(19) United States
(12) Patent Application Publication
(10) Pub. No.: US 2009/0156632 A1
(43) Pub. Date: Jun. 18, 2009

(54) DOSAGE REGIMEN FOR PRASUGREL

(76) Inventors: John Thomas Brandt, Indianapolis, IN (US); Nagy Alphonse Farid, Lebanon, IN (US); Joseph Anthony Jakubowski, Indianapolis, IN (US); Christopher David Payne, Hampshire (GB); Govinda Jayanath Weerakkody, Carmel, IN (US); Kenneth John Winters, Memphis, TN (US)

Correspondence Address: ELI LILLY & COMPANY, PATENT DIVISION, P.O. BOX 6288, INDIANAPOLIS, IN, 46206-6288, US

(21) Appl. No.: 11/916,817

(22) PCT Filed: Jun. 13, 2006
(86) PCT No.: PCT/US06/23006

§ 371 Date: Aug. 5, 2008

Related U.S. Application Data
(60) Provisional application No. 60/691,740, filed on Jun. 17, 2005.

(51) Int. Cl. A61K 31/4365; A61P 9/00

(52) U.S. Cl. 514/301

(57) ABSTRACT

A dosage regimen for treating vascular disease in a human comprising the steps of administering a loading dosage of about 30 mg to 70 mg of loading dose of prasugrel or a pharmaceutically acceptable salt thereof, and thereafter administering a daily dosage regimen of about 7.5 mg to 15 mg maintenance dose of prasugrel or a pharmaceutically acceptable salt thereof.

### FIELD OF THE INVENTION

The present invention relates to a dosage regimen for the administration of prasugrel to a patient in need thereof.

### BACKGROUND OF THE INVENTION

Vascular disease including myocardial infarction and ischemic stroke is a leading cause of death and disability. While the processes causing vascular disease(s) are complex and not completely understood, an underlying etiology common to the numerous theories includes atherosclerosis due to atherosclerotic lesion formation and the disruption of plaques leading to thrombosis or thromboembolisms.

2-Acetoxy-5-(α-cyclopropylcarbonyl-2-fluorobenzyl)-4,5,6,7-tetrahydrothieno[3,2-c]pyridine (prasugrel), an adenosine diphosphate (ADP) receptor antagonist, is a potent inhibitor of ADP-mediated platelet aggregation in vivo. U.S. Pat. No. 5,288,726 discloses tetrahydrothienopyridine derivatives including prasugrel. U.S. Pat. No. 6,693,115 B2 discloses acid addition salts of prasugrel. US Patent publication 2004/0024013 A1 discloses a method of treating vascular diseases by administering prasugrel or a pharmaceutically acceptable salt thereof, and aspirin. U.S. Pat. No. 6,693,115 B2 also discloses a unit dosage regimen for oral administration comprising administering from 0.1 mg (preferably 1 mg) to 1000 mg (preferably 500 mg) of acid addition salts of prasugrel. The suggested dosage regimen further comprised administering the above unit dose from 1 to 7 times per day.

The dosage regimen of the invention (1) provides maximal benefit, (2) minimizes safety and/or adverse events and/or (3) minimize non-response issues.

### SUMMARY OF THE INVENTION

The present invention provides a method of treating vascular diseases in a human comprising the steps of:

(a) administering a loading dose of about 30 mg to 70 mg of a compound of formula (I)

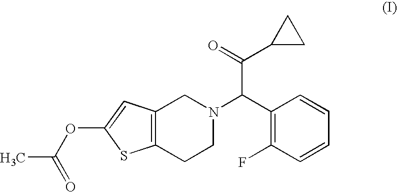

or an equivalent amount of a pharmaceutically acceptable salt thereof; and thereafter

(b) administering a maintenance dose of about 7.5 mg to 15 mg of a compound of formula (I) or an equivalent amount of a pharmaceutically acceptable salt thereof.

The present invention also provides a dosage regimen for the administration of the compound of formula (I) or a pharmaceutically acceptable salt, solvate, active metabolite, prodrug, racemate or enantiomer thereof, wherein the thrombus formation-induced or an embolization-induced disease is an acute coronary syndrome.

The present invention also provides a dosage regimen for the administration of the compound of formula (I) or a pharmaceutically acceptable salt, solvate, active metabolite, prodrug, racemate or enantiomer thereof for the treatment of thrombus formation-induced or an embolization-induced disease wherein the thrombus formation-induced or an embolization-induced disease is an acute coronary syndrome treated in conjunction with a coronary intervention procedure such as percutaneous coronary intervention (PCI) procedure, including intracoronary stent placement.

The present invention also provides a dosage regimen for use of prasugrel in patients undergoing PCI in the absence of acute coronary syndrome or in patients undergoing other vascular interventions (e.g., carotid artery stenting, renal artery stenting, peripheral arterial stenting)

The present invention also provides a dosage regimen for use of the HCl salt of the compound of formula (I) comprising administering 40 to 60 mg base equivalent of said HCl salt as a loading dose followed at an appropriate interval by administering a maintenance dose of 10 to 15 mg base equivalent of prasugrel HCl.

In another embodiment, the present invention also provides dosage regimen wherein the loading dose is equivalent to 40 mg of the compound of formula (I) and the daily maintenance dose is equivalent to 10 mg of the compound of formula (I).

The present invention also provides a dosage regimen wherein the loading dose is equivalent to 60 mg of the compound of formula (I) and the daily maintenance dose is equivalent to 10 mg of the compound of formula (I).

The present invention also provides a dosage regimen wherein the loading dose is equivalent to 40 mg of the compound of formula (I) and the daily maintenance dose is equivalent to 15 mg of the compound of formula (I).

The present invention also provides a dosage regimen comprising administering a maintenance dose of prasugrel HCl equivalent to 15 mg of the compound of formula (I).

The present invention also provides a dosage regimen comprising administering a daily maintenance dose of prasugrel HCl equivalent to 10 mg of the compound of formula (I).

The present invention also provides a dosage regimen for the use of prasugrel HCl for the treatment of acute coronary syndrome in conjunction with percutaneous coronary intervention (PCI) procedures comprising administering a loading dose equivalent to 40 mg to 60 mg of the compound of formula (I) and/or a daily maintenance dose equivalent to 10 mg to 15 mg of the compound of formula (I).

The present invention also provides a dosage regimen for the treatment of a thrombus formation-induced or an embolization-induced stroke or atherothrombotic event in a patient with high-risk vascular disease comprising administering a loading dose equivalent to 40 mg to 60 mg base equivalent of the compound of formula (I) and/or a maintenance dose equivalent to 10 mg to 15 mg of the compound of formula (I).

The present invention relates to a dosage regimen comprising about 40 to 60 mg base equivalent loading dose and/or about 10 to 15 mg daily maintenance dose of a hydrochloric acid addition salt of the compound of formula (I) (prasugrel HCl) singly or in combination with other effective anti-platelet agents for the treatment and/or prevention of vascular diseases, such as aspirin of which the daily dose is about 50 mg to 500 mg and preferably about 75 to 325 mg.

### DEFINITIONS

For the purpose of the present invention the term, “vascular diseases” refers to diseases treatable, preventable, or able to be ameliorated by the use of a thienopyridine, particularly prasugrel. Examples of vascular diseases encompassed by the invention include coronary occlusion, restenosis, acute coronary syndrome (ACS) including ACS with medical management (ACS-MM), high risk vascular diseases (HRVD), cerebro vascular aneurysm (CVA), congestive heart failure, cardiac alternation, ventricular aneurysm, mural aneurysm, myocardial infarction, cardiac arrest, cardiac dysrhythmia including atrial fibrillation, cardiac edema, cardiac dyspnea, cardiac failure, tachycardia, stroke, transient ischemic attack, cardiac hemoptysis, cardiac incompetence, cardiac murmur, cardiac syncope, cardiac tamponade, cerebrovascular disease and/or peripheral artery disease

The term “administering” as used herein is intended to include various routes of administration, particularly oral, which allow for a compound of the invention to perform its intended function of treating and/or preventing the occurrence or recurrence of vascular diseases.

The terms “thrombosis” and “thromboembolism” as used herein bear their common meanings. Thus thrombosis induced or thromboembolism induced diseases are diseases caused by or exacerbated by the presence of or the condition of having or being predisposed to thrombosis or thromboembolism. Examples of such disease include some of the diseases characterized as vascular diseases such as myocardial infarction, angina, stroke, pulmonary embolism, transient ischemic attack, deep vein thrombosis, thrombotic re-occlusion subsequent to a coronary intervention procedure, heart surgery or vascular surgery, peripheral vascular thrombosis, vascular disorders related diabetes mellitus, and/or syndrome X (metabolic syndrome), and heart failure.

The term “treatment” as used herein bears its ordinary meaning and includes the amelioration, inhibition, prevention of occurrence or recurrence, reduction in severity or effect of vascular diseases (including thrombosis induced or thromboembolism induced) such as myocardial infarction, angina, stroke, pulmonary embolism, transient ischemic attack, deep vein thrombosis, thrombotic re-occlusion subsequent to a coronary intervention procedure, heart surgery or vascular surgery, peripheral vascular thrombosis, vascular disorders related diabetes mellitus, and/or syndrome X (metabolic syndrome), vascular diseases associated with diabetes, and heart failure.

Prasugrel is the non-proprietary (generic) name for the compound of formula (I). Chemical abstract Service (CAS) lists prasugrel as covering the base and the HCl salt. Prasugrel is registered at the International Nonproprietary Name (INN), designated by the World Health Organization (WHO), as 5-[(1RS)-2-cyclopropyl-1-(2-fluorophenyl)-2-oxoethyl]4,5,6,7-tetrahydrothieno[3,2-c]pyridin-2-yl acetate. Prasugrel hydrochloride is registered at the United States Adopted Name (USAN) as (±)-2-[2-acetyloxy-6,7-dihydrothieno[3,2-c]pyridin-5(4H)-yl]-1-cyclopropyl-2-(2-fluorophenyl)ethanone hydrochloride. For the purpose of the present invention prasugrel is the base of the compound of formula (I). The present invention relates to observations made using the base of the compound of formula (I) in the course of clinical trials except where specified.

The term “loading dose” as used herein refers to the amount of a compound of formula I necessary, sufficient and/or effective to control, arrest, treat or prevent the escalation of the particular vascular disease in a patient acutely presenting or in imminent danger or condition of presenting with an acute form of the vascular disease such as for example, thrombo-embolism, restenosis, etc. One definition of loading dose refers to the amount of a compound of formula I, its pharmaceutically acceptable salt, solvate, etc., or other platelet aggregation inhibitor administered to a patient from the time of presentation to the time of initiating other procedures such as for example, surgery, precutaneous coronary intervention, or other angioplasty procedures including immediately following such procedures. More generally, a loading dose is the amount of a drug(s) administered to control, arrest or prevent further deterioration in the patient's condition given sometime after presentation but before initiation or completion of an interventional procedure or before the initiation of a maintenance dose. For the purpose of this invention, a reasonable loading dose may be administered over a period from about immediately upon presentation to a qualified caregiver to about 7 days, more preferably from about 1 hour to 3 days after presentation, and most preferably from about 15 minutes after presentation to about one day and may include multiple doses within the period as determined necessary by the treating physician. It is understood, that a treating physician may recommend that a loading dose be taken in fractional amounts for a specified patient or patient population. For example, for a particular patient or patient population a physician may recommend a fractional loading dose of about 20 mg to 30 mg equivalent of the compound of formula I and/or a fractional maintenance dose for special populations or the specific presentations and/or medical history of a particular patient. Similarly, to achieve a loading dose of 40 to 60 mg, a treating physician may recommend two 20 to 30 mg doses taken at appropriate intervals suited to the particular circumstances of a patient. Thus, the present invention contemplates and encompasses the use of fractional loading doses one or more times to achieve the desired loading dose for a particular patient or patient population. Regardless of the method chosen, a loading dose is given before initiation of an adjunct interventional procedure or immediately after such procedure but before initiation of a maintenance dose. Where an interventional procedure is not performed, a loading dose may still be administered to arrest, stabilize or control the situation followed by a maintenance dose administered as necessary to return the patient to a baseline or near baseline condition.

The phrases “about” or “equivalent to” as used herein refer only to molar weight equivalence or chemical equivalence of the compound of formula I given as the acid addition salt, preferably the HCl salt or solvate thereof. For example the phrase “about 60 mg equivalent loading dose of prasugrel or a pharmaceutically acceptable salt thereof” means that the amount of active ingredient selected is calculated based on 60 mg of prasugrel (base) unless otherwise specified. Thus about 65.86 mg of prasugrel HCl is equivalent to 60 mg of prasugrel. Similarly, 10.98 mg dose of prasugrel HCl is equivalent to a 10 mg dose of prasugrel, 16.47 mg dose of prasugrel HCl is equivalent to a 15 mg dose of prasugrel, and 43.91 mg dose of prasugrel HCl is equivalent to a 40 mg dose of prasugrel, by mole ratio (e.g. adjusting for molecular weight differential). The terms “about” and “equivalent to” are not synonymous with the term “bioequivalence. The bioequivalence of a dose of Prasugrel to a given dose of Prasugrel HCl is not the object of the present invention and is neither implied nor inferred herein.

The term “maintenance dose”, as used herein, means the dose administered to a patient following the loading period. The dose is an effective amount to achieve the desired long, medium or short-term results when used as directed and in the absence of other complications. For the purpose of this invention, a reasonable maintenance period (the period of time following the loading period in which the subject is continuously administered a dose of prasugrel or a pharmaceutically acceptable salt thereof at a dosage level lower than the loading dose for the purpose of maintaining a beneficial on-going level of inhibition of platelet aggregation) may be a period of from about 3 days to about 700 days, and preferably from about 7 days to about 365 days. The maintenance dose is preferably administered daily. Where the patient skips a dose or the caregiver recommends adjustment or skipping, it is still preferred that the dose given approximates 10 to 15 mg equivalent of the compound of compound I (prasugrel) per day. It is understood, that a treating physician may recommend that a maintenance dose be taken in fractional amounts for specified patient or patient population. For example, for a particular patient or patient population a physician may recommend a fractional maintenance dose of about 5 mg to 7.5 mg equivalent of the compound of formula I. Similarly, to achieve a maintenance dose of 10 mg per day, a treating physician may recommend two 5 mg doses taken at appropriate intervals suited to the particular circumstances of a patient. Thus, the present invention contemplates and encompasses the use of fractional doses singly or multiply to achieve the desired maintenance dose for a particular patient or patient population. Ultimately, the precise amount, initiation frequency and length of therapy according to this invention is a determination to be made by the treating or attending physician and tailored to the particular patient's needs or history including size, body weight, medical history, predispositions and co-morbidities.

The compound of formula (I) may be administered singly and/or in combination with other “pharmaceutically acceptable carrier” which allows the compound(s) to perform its intended function. Examples of such carriers include solutions, solvents, dispersion media, delay agents, emulsions, micro particles and the like for combination therapies.

The compound of formula (I) may be used in a combination therapy with other effective anti-platelet agents selected from the group consisting of aspirin, clopidogrel, and active metabolites thereof, wherein both treatments are initiated simultaneously or sequentially within a short period (typically within 0 to 30 days) after initiation of the first therapy. The phrase combination therapy also connotes the use of a combination delivery method wherein both chosen therapies are delivered in a single tablet, capsule, inhalation mechanism, intravenous solution or rectal suppository. The period of combination therapy as defined above may be from about 30 days to about 700 days, and preferably from about 30 days to about 365 days. Ultimately, the precise period of therapy according to this invention is a determination to be made by the treating or attending physician and tailored to the particular patient including considerations of presenting co-morbidities.

The compound of formula (I) can form acid addition salts (pharmaceutically acceptable salt). There is no particular restriction on the nature of these salts, provided that, where they are intended for therapeutic use, they are pharmaceutically acceptable. Preferred acid addition salts includes but is not limited to the HCl salt, the HBr salt, tartaric acid (tartrate salt), and the maleic acid addition salt (maleate salt). Most preferred additions salt include the HCl salt and the maleate salt, the HCl salt being particularly preferred.

In some cases, when prasugrel or its pharmaceutically acceptable salts are allowed to stand in contact with the atmosphere or are recrystallized, they may absorb water or may take up water to form a hydrate. The present invention encompasses these hydrates.

### PREFERRED EMBODIMENTS OF THE INVENTION

One embodiment of the present invention is a dosing regime comprising administering a loading dose of 40 to 60 mg equivalent of prasugrel followed at an appropriate interval by a maintenance dose of 7.5 to 15 mg equivalent of prasugrel singly or in combination with 75 to 325 mg of Aspirin to a patient in need thereof.

A more preferred embodiment of the invention is a dosage regimen comprising administering a loading dose of 60 mg equivalent of prasugrel followed at an appropriate interval by a maintenance dose of 7.5 to 10 mg equivalent of prasugrel singly or in combination with 75 to 325 mg of aspirin to a patient in need thereof.

A particularly preferred embodiment of the invention is a dosage regimen comprising administering a loading dose of 40 mg equivalent of prasugrel followed at an appropriate interval by a maintenance dose of 10 mg equivalent of prasugrel in combination with 75 to 325 mg of aspirin to a patient in need thereof.

A particularly preferred embodiment of the invention is a dosage regimen comprising administering a loading dose of 60 mg equivalent of prasugrel followed at an appropriate interval by a maintenance dose of 10 mg equivalent of prasugrel in combination with 75 to 325 mg of aspirin to a patient in need thereof.

A most preferred embodiment of the present invention is a dosing regimen comprising administering the prasugrel HCl equivalent of 40 or 60 mg loading dose prasugrel followed at an appropriate interval by administering the prasugrel HCl equivalent of 10 mg maintenance dose prasugrel in combination with 325 mg of Aspirin.

In a preferred embodiment, the present invention relates to a dosage regimen of prasugrel that provides optimum therapeutic benefit to patients. Applicants have discovered that patients receiving the preferred dosage regimen were significantly less likely to fail to respond to treatment compared with those undergoing treatment with clopidogrel. Several investigators have reported that about 20 to 30 percent of patients do not respond to clopidogrel therapy or respond inadequately based on an objective application of the current definitions of response and/or non-response to thienopyridines

Applicants have also discovered a dosage regimen comprising a loading dose of prasugrel that provides maximal inhibition of platelet aggregation as measured in an acute setting while minimizing the potential for side effects such as bleeding. The optimum loading doses of prasugrel discovered provide quicker on-set of platelet inhibition, and quicker and higher achievement of maximal inhibition of platelet activation and/or aggregation and provide an optimum therapeutic window for treatment with prasugrel not previously known.

Applicants have also discovered a dosage regimen for prasugrel that provides maximal or optimal on-going (maintenance) inhibition of platelet aggregation for patients needing short or medium to long term treatment or prevention. The dose regimen of the present invention minimizes the potential for a second or recurring ischemic attack or infarction; prevents or minimizes the potential for a first occurrence of an ischemic attack; and provides the above beneficial effects while minimizing the potential for or extent of side effects such as bleeding to manageable or tolerable (in view of the benefits) levels compared to placebo and/or other thienopyridines and/or other oral antiplatelet agents.

### Preparing Compounds of the Invention

Prasugrel HCl may be prepared following procedures disclosed in PCT application WO 02/04461, published Jan. 17, 2002, now U.S. Pat. No. 6,693,115 B2, the entire contents of which are incorporated herein by reference.

### Method of Using the Invention

Preclinical studies involving the base of prasugrel showed that prasugrel was at least 10 times more potent than clopidogrel. These preclinical studies did not provide, indicate or suggest an optimum dose of prasugrel for human use. A subsequent clinical trial showed that compared to the base the HCl salt (prasugrel HCl) provided a statistically significant higher level of bioavailability for patients undergoing concomitant therapy with H1-antagonists such as ranitidine. The applicants then designed a clinical trial to test several doses of prasugrel against the standard regimen for clopidogrel in an effort to determine an optimum dose of prasugrel for administration to a patient in need thereof. The objective was to discover an optimum dose(s) that maximize the efficaciousness in inhibiting platelet aggregation (i.e. prevention or treatment of a patient suffering from or susceptible to platelet aggregation and diseases related thereto) while simultaneously minimizing risks related to thienopyridine therapy such as, for example, bleeding. In the course of clinical trials the applicants conceived of and reduced to practice an optimum human dose regimen that provides maximal platelet aggregation compared to clopidogrel as well as an optimum loading dose that when combined with an optimum maintenance dose provided improved treatment outcomes in conjunction with percutaneous coronary intervention for patients having acute coronary syndromes. The improved benefits observed for the optimum dose were evident whether the patients were undergoing concomitant aspirin therapy or not. Applicants' also discovered a dosage regimen that provides an optimum maintenance dose for patients needing to prevent or minimize the potential of a recurrence of thrombosis or thromboembolism such as, for example, ischemic attack or myocardial infarction.

The combination of the dosage regimen of prasugrel herein and aspirin for the purpose of practicing the invention may be accomplished by having individual or unit doses of the compound of formula I and aspirin (i.e. separate containers) or by having a combined prepackaged or pre-formulated dose of aspirin and the compound of formula I.

The specific loading and maintenance doses of prasugrel administered to obtain therapeutic or prophylactic effect within the identified advantageous limits of this invention will of course be determined by the particular circumstances of the patient, including, for example, the route of administration and the particular vascular disease being treated. A preferred dosing regimen comprises a loading dose from about 40 to about 60 mg of prasugrel followed at an appropriate interval by administration of a maintenance dose comprising about 10 to 15 mg of prasugrel one or two times per day or as recommended by the treating physician. For non-acute presentations i.e. for patient needing maintenance therapy, a preferred dosage regimen comprises a maintenance dose of prasugrel HCl equivalent to about 10 mg of prasugrel. The amount of loading and/or maintenance doses and the frequency of dosing and length of dosing using the physician-selected dosing regimens are determinations to be made by the treating physician(s) to achieve maximum efficacy for the particular patient and circumstance including considerations of age, weight, particular vascular disease presented, co-morbidities, among other customary and proper considerations.

The co-administration of aspirin in combination or conjunction with a compound of the invention to obtain therapeutic or prophylactic effect will of course be determined by the particular circumstances of the patient. In general the amount of aspirin for the purpose of the present invention is about that generally approved for the particular patient population, e.g. from about 75 mg to about 325 mg of aspirin 1 to 3 times daily.

### EXAMPLES

Methods of preparing compounds of the invention were published and therefore known to one of skill in the art. Such methods disclosed in for example U.S. Pat. No. 5,288,726 and U.S. Pat. No. 6,693,115 B2 the contents of which are incorporated herein by reference.

The following formulation examples, and test examples are intended to further illustrate the present invention and are not intended to limit the scope of this invention.

### Formulation Example 1

Prasugrel HCl (10.98 mg/tablet equivalent to 10 mg/tablet base), mannitol, hydroxypropyl methylcellulose, croscarmellose sodium, microcrystalline cellulose and magnesium stearate are blended and then roller compacted to produce a granulation. To the resulting granulation, additional croscarmellose sodium, microcrystalline cellulose and magnesium stearate are added and the material is blended and compressed to form tablets weighing 250 mg. An Opadry® II beige film coating mixture is added to water and then sprayed onto these tablets in a side vented coating pan.

### Clinical Example 1

A Comparative Study of the Effects of Prasugrel and Clopidogrel on Platelet Function in Healthy Subjects
Background: Antiplatelet agents such as aspirin and clopidogrel are effective in the secondary prevention of atherothrombotic events. In preclinical studies prasugrel showed more potent inhibition of platelet aggregation (IPA) than clopidogrel. This study examined the tolerability, safety, and IPA profile of prasugrel compared with clopidogrel.
Method: A double-blind, placebo-controlled, multiple-dose study of healthy male volunteers randomized into 5 groups (n=6): prasugrel (5, 10 and 20 mg), clopidogrel (75 mg), and placebo. Study medications were taken once daily for 10 days. Platelet aggregation induced by 20 μM ADP was measured turbidometrically at selected intervals.
Result: Multiple oral dosing of prasugrel was well tolerated at doses of 5 to 20 mg for 10 days. For median maximum bleeding times, there were no significant differences (p≧0.05) between the active treatments. Twenty-four hours after the final dose, prasugrel dose-dependently increased IPA (39.2%-68.3%), an effect for all prasugrel doses greater than that observed with clopidogrel (15.7%; p<0.05). Furthermore, 2 of 6 clopidogrel-treated subjects demonstrated minimal IPA at Day 10. See Table 1.

TABLE 1
Levels of IPA assessed using 20 μM ADP 24 hours after 10^{th} dose

| Mean ± | | | | | Clop- |
|---|---|---|---|---|---|
| SE | | Prasugrel | | | idogrel |
| (n = 6) | Placebo | 5 mg | 10 mg | 20 mg | 75 mg |
| IPA | 9.2 ± 4.0 | 39.2 ± 4.4 | 58.2 ± 4.9 | 68.3 ± 5.4 | 15.7 ± 6.8 |
| (%) | | | | | |
| p-value | — | <0.001 | <0.001 | <0.001 | <0.001 |
| vs. | | | | | |
| Placebo | | | | | |
| p-value | <0.001 | 0.011 | <0.001 | <0.001 | — |
| vs. | | | | | |
| Clop- | | | | | |
| idogrel | | | | | |
| 75 mg | | | | | |

Conclusion:
Prasugrel was well tolerated when administered to healthy subjects as 10 daily doses of up to 20 mg. Platelet aggregation at Day 10 in all prasugrel-treated subjects in all dose groups was consistently decreased contrasting the IPA observed with clopidogrel (75 mg).

### Clinical Example 2

Prasugrel Achieves Significantly Higher Inhibition of Platelet Aggregation and a Lower Rate of Non-Responders Compared with Clopidogrel in Aspirin-Treated Patients with Atherosclerotic Vascular Disease
Background: Lower levels of inhibition of platelet aggregation (IPA) with clopidogrel increase the risk of thrombotic events. This study analyzed IPA and non-responder rates with prasugrel (Pras), a novel P2Y₁₂ antagonist, vs. clopidogrel (Clop) in aspirin-treated patients.
Methods: After 7-days on aspirin 325 mg, 101 subjects were randomized to 1 of 5 dosing regimens, a loading dose (LD) on day 1 and a daily maintenance dose (MD) on days 2-28: prasugrel—40 mg LD/5 mg MD, 40 mg LD/7.5 mg MD, 60 mg LD/10 mg MD, or 60 mg LD/15 mg MD or clopidogrel—300 mg LD/75 mg MD). IPA to 20 μM ADP was measured by turbidometric aggregometry. Non-responders were defined as those not achieving ≧20% IPA at 4 h after LD and at pre-dose during MD.
Results: At 4 h after LD, IPA with prasugrel 40 mg (60.6%) and 60 mg (68.4%) was higher than with clopidogrel 300 mg (30.0%, p<0.0001). At day 28, higher pre-dose IPA was also observed with prasugrel 10 mg (57.5%) and 15 mg (65.8%) vs. clopidogrel 75 mg (31.2%) (p<0.0001). Non-responder rates with clopidogrel LD (52%) and clopidogrel MD (46%) were markedly greater than either prasugrel LD or prasugrel 10 and 15 mg MD (see FIG. 1).
Conclusions: In aspirin-treated patients, prasugrel LD of 40 or 60 mg and MD of 10 or 15 mg achieve higher IPA and a lower rate of non-responders compared to standard clopidogrel LD and MD.
   The above data show that applicants have discovered a beneficial and superior dosing regimen whcih minimizes the potential for adminsitering doses that are non-responsive to patients undergoing treatment with prasugrel (emphasis added).

### Clinical Example 3

Clopidogrel Responders and Nonresponders: A Comparison with Prasugrel a Novel Thienopyridine P2Y₁₂ Receptor Antagonist
Background: Lower levels of inhibition of platelet aggregation (IPA) with clopidogrel have been associated with a higher risk of adverse events after percutaneous coronary intervention (PCI). Thresholds of IPA, objectively defined with Bayesian classification theory, were used to identify responders and non-responders to clopidogrel and prasugrel, a novel thienopyridine P2Y₁₂ receptor antagonist.
Methods: An integrated database of ADP (5 μM and 20 μM)-induced platelet aggregation as measured by turbidometric aggregometry was analyzed from three single-center cross-over clinical pharmacology studies. Subjects (N=112, aged 18-65 years) were healthy volunteers with a baseline maximum platelet aggregation (MPA) of >70% to 20 μM ADP and were randomized to either a clopidogrel 300 mg loading dose (LD) or a prasugrel 60 mg LD. The change in MPA (ΔMPA) from baseline and IPA were evaluated at both 4-5 and 24 hours after the LD. A responder was defined as an individual achieving either a ΔMPA≧15% or an IPA≧20% in response to 20 μM ADP. For 5 μM ADP, a responder was defined by a ΔMPA≧20% or an IPA≧25%.
Results: For 5 μM ADP, approximately 75% of subjects were responders to clopidogrel and 100% were responders to prasugrel (p<0.001). For 20 μM ADP, 60% were responders to clopidogrel and 100% were responders to prasugrel (p<0.001, Table 2).

TABLE 2
Number and Percentage of Responders

| | | |
|---|---|---|
| Clopidogrel | Prasugrel | |
| (N = 112) | (N = 91) | P-value |

5 μM ADP

| | | | |
|---|---|---|---|
| ΔMPA ≧ 20% | 82 (73%) | 91 (100%) | <0.001 |
| IPA ≧ 25% | 87 (78%) | 91 (100%) | <0.001 |

20 μM ADP

| | | | |
|---|---|---|---|
| ΔMPA ≧ 15% | 68 (61%) | 91 (100%) | <0.001 |
| IPA ≧ 20% | 64 (57%) | 91 (100%) | <0.001 |

Conclusions: The proportion of responders and non-responders depends in part on the concentration of ADP. The response rate to a prasugrel 60 mg LD was significantly higher than that observed with a clopidogrel 300 mg LD, as measured with thresholds objectively defined with Bayesian classification theory.

1. A method for treating vascular disease in a human comprising the steps of:
(a) administering a loading dose of about 30 mg to 70 mg of a compound of formula (I) 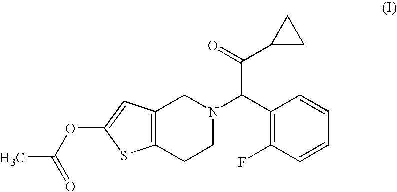
or an equivalent amount of a pharmaceutically acceptable salt thereof; and thereafter
(b) administering a maintenance dose of about 7.5 mg to 15 mg of a compound of formula (I) or an equivalent amount of a pharmaceutically acceptable salt thereof.

2. The method according to claim 1, wherein the compound is 2-acetoxy-5-(α-cyclopropylcarbonyl-2-fluorobenzyl)-4,5,6,7-tetrahydrothieno[3,2-c]pyridine hydrochloric acid addition salt (prasugrel HCl).

3. The method according to claim 1, wherein the loading dose is equivalent to about 40 mg to 60 mg of the compound of formula (I).

4. The method according to claim 3, wherein the loading dose is equivalent to 40 mg of the compound of formula (I) and the maintenance dose is equivalent to 10 mg of the compound of formula (I) administered daily.

5. The method according to claim 3, wherein the loading dose is equivalent to 60 mg of the compound of formula (I) and the daily maintenance dose is equivalent to 10 mg of the compound of formula (I).

6. The method according to claim 3, wherein the loading dose is equivalent to 60 mg of the compound of formula (I) and the daily maintenance dose is equivalent to 15 mg of the compound of formula (I).

7. The method according to claim 3, wherein the loading dose is equivalent to 40 mg of the compound of formula (I) and the daily maintenance dose is equivalent to 15 mg of the compound of formula (I).

8. The method according to claim 1 wherein the patient is further administered a daily dose of about 75 mg to 325 mg of aspirin.

9. A method for preventing and/or treating thrombosis induced or a thromboembolism induced disease in a human comprising administering a daily maintenance dose of about 7.5 mg to 15 mg of the compound of formula (I) 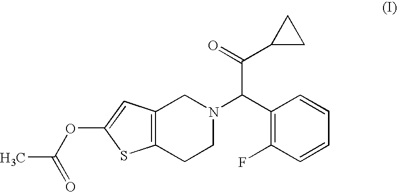
or a pharmaceutically acceptable salt thereof.

10. The method according to claim 9 wherein 2-acetoxy-5-(α-cyclopropylcarbonyl-2-fluorobenzyl)-4,5,6,7-tetrahydrothieno[3,2-c]pyridine (prasugrel) is administered as its HCl salt.

11. The method according to claim 1, wherein the vascular disease is acute coronary syndrome, medically managed acute coronary syndrome, stroke, HRVD, each optionally treated in conjunction with percutaneous coronary intervention procedures and/or aspirin.

12. A method for prevention and/or treatment of a thrombosis induced or a thromboembolism induced disease in a human comprising administering a daily maintenance dose of about 7.5 mg to 10 mg of prasugrel.

13. The method according to claim 2, wherein the loading dose is 43.91 mg of prasugrel hydrochloride and the daily maintenance dose is 10.98 mg of prasugrel hydrochloride.

14. The method according to claim 2, wherein the loading dose is 65.86 mg of prasugrel hydrochloride and the daily maintenance dose is 10.98 mg of prasugrel hydrochloride.

15. The method according to claim 2, wherein the loading dose is 43.91 mg of prasugrel hydrochloride and the daily maintenance dose is 10.98 mg of prasugrel hydrochloride.

16. (canceled)

17. The method according to claim 2, wherein the loading dose is equivalent to about 40 mg to 60 mg of the compound of formula (I).

18. The method according to claim 2 wherein the patient is further administered a daily dose of about 75 mg to 325 mg of aspirin.
